(19) United States
(12) Patent Application Publication
(10) Pub. No.: US 2023/0348344 A1
(43) Pub. Date: Nov. 2, 2023

(54) SEPARATION OF CARBON DIOXIDE AND SULFUROUS MATERIALS FROM GASEOUS MIXTURES

(71) Applicant: 8 Rivers Capital, LLC, Durham, NC (US)

(72) Inventor: Navid Rafati, Durham, NC (US)

(21) Appl. No.: 18/016,961

(22) PCT Filed: Jul. 21, 2021
(86) PCT No.: PCT/IB2021/056607

§ 371 Date: Jan. 19, 2023

Related U.S. Application Data
(60) Provisional application No. 63/054,974, filed on Jul. 22, 2020.

(51) Int. Cl. C07C 7/06; C10L 3/10; B01D 53/00; B01D 3/14; B01D 3/36

(52) U.S. Cl. CPC C07C 7/06; C10L 3/103; B01D 53/002; B01D 3/143; B01D 3/36; C10L 2290/543

(57) ABSTRACT

The present disclosure relates to systems and methods useful in the separation of a mixed gaseous stream into one or more individual components. Resulting products can include, for example, carbon dioxide, sulfurous compounds (e.g., hydrogen sulfide), nitrogen, helium, fuel gas (e.g., natural gas, or a single or mixed hydrocarbon stream), and liquefied natural gas. The methods can include processing within a first contacting column a combination of a multi-component feed stream and an anti-freezing agent, removing from the first contacting column a stream containing a fuel gas, removing from the first contacting column a stream containing a sulfurous material, and processing the stream containing the sulfurous material in a second contacting column to provide a stream comprising at least ethane and to provide a separate stream comprising at least a portion of the sulfurous material.

### FIELD OF THE INVENTION

The present disclosure relates to separation of acidic gases from a mixed hydrocarbon stream. Separation can be carried out utilizing systems and methods whereby the mixed stream is cooled down and fractionated in consecutive distillation columns with the help of liquid agents.

### BACKGROUND

Hydrocarbon gas streams are often contaminated with one or more further components, such as sulfur compounds (e.g., hydrogen sulfide—or H₂S) and carbon dioxide (CO₂). Natural gas, associated petroleum gas (APG), and refinery off-gas are examples of gas streams that can include methane (CH₄—or “C₁”), as well as significant quantities of longer chain hydrocarbons, such as ethane (C₂H₆—or “C₂”) and even higher carbon number hydrocarbons, including C₃ and greater hydrocarbons. These higher number hydrocarbons can be separated to provide valuable hydrocarbon streams, particularly upon removal of contaminants, such as hydrogen sulfide and carbon dioxide. As an example, a sour natural gas stream can have an approximate composition as follows (on a molar, water-free basis): 62% CH₄; 7% C₂H₆; 3% C₃H₈; 3% C₄₊; 20% H₂S; and 5% CO₂.

Current methods of removing H₂S and CO₂ from mixed gas streams, such as natural gas streams, include amine scrubbing; membrane treatment; adsorption on solid adsorbents; processing in low temperature separation systems such as the Ryan-Holmes process (see U.S. Pat. Nos. 4,318,723 and 4,462,814), the Controlled-Freeze Zone™ process (see U.S. Pat. No. 8,312,738), and the processes described in U.S. Pat. No. 9,945,605; and hybrid processes that use a combination of any aforementioned methods, such as the hybrid processes described in U.S. Pat. No. 8,955,354. These processes all involve high capital investments and high ongoing operating costs and inevitably lead to significant discharge of CO₂ to the atmosphere from the energy systems associated with them.

In particular, the method described by Holmes et al. is a cost-effective method to separate methane from natural gas streams containing relatively large amount of carbon dioxide using distillation methods. In such methods, the separation of methane from other components of a hydrocarbon mixture including carbon dioxide and hydrogen sulfide is carried out in a distillation column (which may be referred as a “de-methanizer”) wherein the top section of the column is maintained at temperatures whereby relatively pure methane product can be obtained as a distillate. Such a temperature range normally results in the solidification of acidic components, including carbon dioxide, but the use of a liquid agent in such a distillation column can effectively suppress the formation of solid acidic compounds at such temperatures.

The liquid agent preferably comprises a C4-C6 alkane, such as butane, pentane, hexane, or a mixture of such alkanes and is preferably introduced into the distillation column at a stage near the top of the column or mixed with the reflux stream (i.e., at locations where the highest likelihood of solid particle formation exist). The bottom product from such a distillation column generally contains all other constituents of the feed stream including C2+ hydrocarbons, CO₂, H₂S, and other sulfurous materials.

The methods described by Holmes et al. include shortcomings, however, and these are particularly evident in relation to the processing of a sour feedstock than contains H₂S and C2 in addition to at least C1 and CO₂. When available, H₂S and C2 normally leave the bottom of the de-methanizer combined with CO₂, C3+hydrocarbons, and other sulfur species. Ethane is a valuable feedstock to the petrochemical industry, and it is can be desirable to recover ethane from the de-methanizer bottoms. It can be quite challenging, however, to use distillation for separation of the de-methanizer bottoms in a Ryan-Holmes process in the presence of H₂S given the complex and intense interactions between various molecule pairs in such a mixture. Hydrogen sulfide forms an azeotrope with both ethane and propane, which makes their complete separation using conventional distillation methods impractical. Additionally, CO₂ and H₂S have a low relative volatility, which increases the energy consumption for their distillative separation. Alternatives have been proposed to overcome such limitation, but known alternatives generally are not economically attractive. These alternatives focus on selective separation of H₂S prior to or after Ryan-Holmes processing, typically using chemical or physical solvents. These hybrid alternatives result in a substantial increase in the capital investment and operating cost of the processing plant, and they typically disfavor the use of Ryan-Holmes for the processing of feedstock with a high H₂S content.

It has been seen that most acidic natural gas streams that have high concentrations of H₂S tend to have relatively high concentrations of ethane as well, which necessitates the recovery of ethane from such a source to improve the economics of the processing facility. Therefore, there remains a need in the art for lower cost, simpler processes that have reduced capital and operating cost for the fractionation of acidic gas streams containing high concentrations of H₂S and CO₂.

### SUMMARY OF THE INVENTION

The present disclosure relates to systems and methods useful in the separation of carbon dioxide and sulfurous compounds from a mixed gaseous stream. The methods and systems described in this disclosure can also be used to remove other inert gases (e.g., nitrogen and helium) from a mixed gaseous stream and generate a substantially pure fuel gas stream that is suitable for direct use as the fuel source or for liquefaction and transportation in liquid form. The systems and methods particularly can be adapted to or configured to contact the mixed gaseous steam comprising the sulfurous material with a liquid agent to improve the separation.

In one or more embodiments, the present disclosure can provide systems and methods whereby an acidic natural gas (or another mixed gas stream) is fractioned to provide one or more components as a product stream. Fractionation can be effective to separate out components such as acidic components (e.g., CO₂ and H₂S ), hydrocarbons (e.g., methane, ethane, propane, and/or higher hydrocarbons, any of which may be in straight or branched chain forms) and inert gases (e.g., nitrogen and helium). The CO₂ stream preferably can be made available for enhanced oil recovery. The C₂ and C3+ hydrocarbons preferably can be separated with H₂S content (or other sulfurous material content) below about 2 ppmw (parts per million based on weight), although higher H₂S contents are not excluded if ultra-low sulfur content is not required. The H₂S (or other sulfurous material) preferably can be separated as a concentrated stream for use as a feed stream to a Claus sulfur production plant, as feed to a sulfuric acid plant, and/or other suitable uses. The H₂S containing stream or other sulfurous material preferably can be produced at high pressure for sequestration in a geologic formation such as a depleted gas or oil well. The natural gas stream (or other gas stream purified of at least a sulfurous material) preferably can have a sulfur content (such as an H₂S content) below about 4 ppm (or even below about 2 ppm, in some embodiments) and a CO₂ content below 50 ppm (or ppmv), which is suitable for nitrogen rejection, helium recovery, and/or further cooling and liquefaction for transportation as liquefied natural gas (LNG).

In one or more embodiments, the present disclosure relates to systems and methods for sequential separation and fractionation of multi-component feed streams, particularly separating CO₂ and sulfurous materials therefrom, utilizing liquid streams. The present disclosure provides a uniquely simple and economic process to separate, for example, acidic impurities such as carbon dioxide and sulfur compounds from fuel gas streams and/or other multi-component feed streams. The presently disclosed systems and methods are particularly useful when a relatively large amount of carbon dioxide and hydrogen sulfide are present within the gaseous stream, and when a carbon dioxide source and a hydrocarbon source are readily available. The hydrocarbon source may particularly be a source of low carbon number hydrocarbons, such as C8 or less, C7 or less, or C6 or less. In particular embodiments, a low carbon number hydrocarbon source may be in any one or more of the following ranges: C3 to C8; C4 to C8; C4 to C7; C5 to C7; or C5 to C6. The use of linear hydrocarbons particularly can be useful for the separation of carbon dioxide from the multi-component stream in a mass-transfer contacting device whereby the complete separation of carbon dioxide from the mixture requires cold temperatures below the triple point of pure carbon dioxide (i.e., −56.4° C. at 5.2 bar), which would normally cause solidification of the carbon dioxide and associated operational problems. For example, the complete separation of methane from carbon dioxide and hydrogen sulfide using a distillation method requires a cryogenic temperature of approximately −95° C. (at a pressure of about 30 bar) at the top of distillation column (with other temperature and pressure ranges being capable of being determined by those of skill in the art). Since carbon dioxide would be expected to be in a solid state at such a low temperature, significant operational problems would occur and lead to blockage of the equipment. The addition of a liquid hydrocarbon stream (e.g., a linear C4-C6 alkane) to such a distillation column at a suitable injection point can effectively suppress the freezing of acidic components within the column and allow for obtaining a relatively pure methane stream as the distillate product. Although linear alkanes can be preferred, branched hydrocarbons that are liquid at useful temperature ranges noted herein also may be utilized.

The present systems and methods are configured to generate a purified and substantially clean carbonaceous material (e.g., a fuel gas) stream from a multi-component feed stream including carbon dioxide and sulfurous material using refrigeration and fractionation. For example, a multi-component feed stream may comprise at least methane, ethane, carbon dioxide and sulfurous compounds, and this multi-component stream can be dried and cooled down to a temperature that is relatively low (e.g., below about 0° C.) but is warmer than the CO₂ triple point temperature. For example, the temperature may be in a range of about −50° C. to about 0° C., about −40° C. to about −20° C., or approximately −30° C. (e.g., −30° C.+/−2° C., 1° C., or 0.5° C.). Thereafter, the multi-component stream can be treated in a series of distillation columns (e.g., two or more) to separate the multi-component stream into a plurality of product streams including one or more of methane, CO₂, H₂S, C2, C3, and C4+ hydrocarbons. A product stream may include only one of the above noted materials as relatively pure product (e.g., 99%, 99.5%, or 99.9% pure by weight) or may comprise a mixture of two, three, or four of the noted materials.

In an example embodiment, the dried and cooled multi-component feed stream can be treated in a first distillation column wherein a liquid stream of linear hydrocarbons is also introduced into the first distillation column from a top section. Exiting the first distillation column can be a distillate stream concentrated in methane and a bottoms stream concentrated in CO₂, sulfurous materials, and C230 hydrocarbons. Because the top product from the first distillation column can be methane (C1), the first distillation column can be referenced as a “De-C1” column The bottom products stream enriched in CO₂ and sulfur can be further treated in a second distillation column (which can be referenced as a “De-C2” column) using a liquid CO₂ stream as the scrubbing agent to generate a substantially or completely sulfur-free distillate stream enriched in ethane (C2) and CO₂ and a bottom product enriched in sulfur and C3 + hydrocarbons. Liquid carbon dioxide used as the scrubbing agent can originate from a carbon dioxide containing stream within the integrated process or from an external source, and the carbon dioxide can be substantially pure or may contain one or more contaminants Similarly, liquid linear hydrocarbons used to suppress the freezing point of the mixture containing CO₂ can originate from the multi-component feed stream or from an external source and preferably is concentrated in alkanes with 4 to 6 carbon chain length, although other hydrocarbon materials as otherwise described herein may be used alternatively, or additionally. The distillate from the De-C2 column can be fractionated into substantially pure CO₂ and ethane streams in a third distillation column (“De-CO₂”) using extractive distillation. Ethane and carbon dioxide form an azeotrope, and the use of a liquid agent can help to prevent the formation of such azeotrope as well as facilitate their distillative separation. Such liquid agent preferably comprises a linear C4-C6 alkane or any other hydrocarbon(s) as already noted above. By doing so, a relatively pure CO₂ distillate and a hydrocarbon mixture with minimal CO₂ content can be obtained from the top and bottom of the De-CO₂ column, respectively. The bottoms from the De-CO₂ column can be conveniently fractionated in a fourth distillation column (i.e., a solvent recovery column) to provide high purity ethane and a hydrocarbon blend stream (azeotrope breaker blend).

In further embodiments, a mixture of at least C2 hydrocarbons and carbon dioxide can be separated into relatively pure constituents using separation techniques other than distillation, for example, a carbon dioxide membrane. This can be done in variety of ways. For example, a two-stage carbon dioxide membrane can be used to obtain a substantially pure ethane product and a carbon dioxide product with relatively low hydrocarbon contamination. Alternatively, a single-stage carbon dioxide membrane can be used, and the low-pressure permeate stream from the single-stage carbon dioxide membrane can be further compressed, refrigerated, and purified in a carbon dioxide stripper column In such column, a relatively high-purity carbon dioxide stream can be obtained as a liquid bottoms product, and the distillate can be mixed with a fuel gas stream or can be recycled to the inlet of membrane.

In some embodiments, the bottoms from the De-C2 column can be additionally processed to obtain a relatively pure H₂S stream and a hydrocarbon blend with low sulfur contamination. For example, when C3+ hydrocarbons are available in the multi-component feed stream, the bottoms from the De-C2 column can be processed in a fifth distillation column (i.e., a “De-H₂S” column) to obtain relatively pure H₂S and C3+ blend with minimum sulfur contamination as the distillate and bottoms products, respectively. Such a separation using a distillation method requires special attention to the azeotrope between propane and hydrogen sulfide. In such a case, a liquid agent can be introduced onto an appropriate stage of the distillation column and avoid the formation of azeotrope between C3 and hydrogen sulfide and minimize the hydrocarbon loss with the distillate. Such liquid agent preferably comprises a linear C4-C6 alkane or any other hydrocarbon(s) as described herein that can effectively break the azeotrope between propane and hydrogen sulfide. By choosing the proper operating conditions it is possible to obtain an overhead distillate that is at least 90% and preferably at least 95% concentrated in H₂S.

The bottoms from the De-H₂S column can contain a majority of any C3+ hydrocarbons in the multi-component feed stream and typically contains less than 500 ppmw (parts per million by weight), and preferably less than 350 ppmw, H₂S. This stream can be further fractionated similar to fractionation of a light naphtha cut in a conventional oil refinery. For example, it can be fractionated to propane and C4+ streams in a conventional de-propanizer or to an LPG (liquefied petroleum gas) blend and C5+ blend in a conventional LPG column.

The multi-component feed stream may also contain sulfurous contents other than H₂S that may have a boiling point similar to or relatively different from H₂S. Additionally, one or more particular types of dehydration adsorbents may result in the formation of COS through the reaction of carbon dioxide and H₂S in the presence of the adsorbent. The reaction can be described as CO₂+H₂S=COS+H₂O. In such a case, these non-H₂S sulfurous compounds may not be completely collected with the H₂S stream (e.g., depending on their relative boiling points to that of H₂S), and all or parts of them may be mixed with the C3+ blend exiting the bottom of the De-H₂S column In such a case, depending on required specification of the C3+ hydrocarbon products, additional processing of the De-H₂S bottoms may be required prior to or after further downstream fractionation. For example, a proper regenerative or non-regenerative molecular sieve adsorbent bed can be used to remove non-H₂S sulfur compounds (e.g., carbon disulfide and mercaptans) from the C3+ stream. As another non-limiting example, a caustic solution can be used to selectively remove residual sulfurous compounds from a liquid C3+ stream.

In cases where COS is the dominant non-H₂S sulfur specie in the multi-component feed stream, a catalytic COS hydrolysis reactor can be embedded within the process at a suitable location. Such a reactor is packed with a suitable catalyst that is active towards the following reaction allowing to convert COS to H₂S which can be separated downstream in De-H₂S column:

COS+H₂O→H₂S+CO₂

A properly designed COS hydrolysis bed can yield outlet COS content of less than 10 ppmv, and preferably less than 5 ppmv. The reaction is preferably carried out at a temperature above the dew point of water at a given pressure in the presence of steam. If sufficient steam content is not present in the reactor feed stream, additional steam can be introduced into the reactor using a suitable method.

In one or more embodiments, the present disclosure alternatively, or additionally, can provide systems and methods for separation of permanent gases, such as helium and nitrogen, from a substantially CO₂-free and/or sulfur-free multicomponent feed stream. For example, the separation of nitrogen from a raw natural gas mixture may be required in order to meet required heating value on final fuel gas. Additionally, removal of helium may be financially attractive given the high market value of this gas. The separation of nitrogen and/or helium from impure natural gas streams is typically carried out by cryogenic distillation at large-scale. Given the very low boiling point of these gases, it is necessary to remove impurities such as CO₂ and sulfurous material from a methane stream prior to cooling the gas so as to avoid solidification of such compounds. The present disclosure provides a cost-effective method to separate helium and or nitrogen from an impure natural gas stream because the distillate from the De-C1 column is essentially condensate-free, sulfur-free, and CO₂-free and can be readily obtained at a low temperature (e.g., in the range of approximately −70° C. to −95° C., depending upon the pressure and, optionally, other operating parameters). Thus, the additional energy expenditure required to separate nitrogen and helium by distillation will be relatively low.

In one or more embodiments, the present disclosure relates to systems and methods for efficient production of liquefied natural gas (LNG) from contaminated natural gas streams. Natural gas is liquefied at a temperature of approximately −160° C., which requires the removal of any impurities that would solidify at such a low temperature prior to cooling the gas. Such impurities in an impure natural gas stream can include, for example, CO₂ and sulfurous compounds. The distillate from the De-C1 column discussed above is essentially condensate-free, sulfur-free, and CO₂-free and can be readily obtained at a low temperature (e.g., in the range of approximately −70° C. to −95° C.), which reduces the amount of additional cooling required to obtain LNG.

In one or more embodiments, systems described herein can utilize a variety of refrigeration methods capable of generating sufficiently cold temperatures as may be preferred according to the present disclosure. For example, a mixed refrigerant comprised of various constituents suitable to generate cold temperatures required by this system upon compression/expansion can be used. Alternatively, a cascade refrigeration system can be used to generate a plurality of different refrigeration temperature levels. For example, a two-stage ethane/propane cascade refrigeration system can generate refrigeration temperatures down to about −40° C. on the C3 refrigerant loop and down to about −95° C. on the C2 refrigerant loop, which can be used to run various low-temperature heat exchangers within the system. Additionally, a methane loop can be added to such cascade system to generate refrigeration temperatures of around −155° C. (and preferably around −165° C.), which may be particularly useful, for example for natural gas liquefaction.

In one or more embodiments, the present disclosure can provide methods for separating a sulfurous material from a multi-component feed stream. In an example embodiment, such methods can comprise: processing within a first contacting column a combination of: 1) a multi-component feed stream including at least a sulfurous material and a fuel gas; and 2) an anti-freeze agent that is liquid at temperatures within a range of about −95° C. to about 0° C.; removing from the first contacting column an overhead vapor stream containing at least a portion of the fuel gas and removing from the first contacting column a bottom product stream containing at least a portion of the sulfurous material; and processing the bottom product stream in a second contacting column to provide a stream comprising at least ethane and to provide a separate stream comprising at least a portion of the sulfurous material. The methods may be further defined in relation to any one or more of the following statements, which are non-limiting of the disclosure and which may be combined in any number and order.

The method further can comprise processing at least a portion of the overhead vapor stream exiting the first contacting column through a unit configured to separate nitrogen from the fuel gas.

The method further can comprise processing at least a portion of the overhead vapor stream exiting the first contacting column through a liquefaction train configured to liquefy at least a portion of the fuel gas and form a liquefied natural gas stream.

The method further can comprise processing at least a portion of the overhead vapor stream exiting the first contacting column through a unit configured to separate helium from the fuel gas.

The processing of the bottom product stream in the second contacting column can include combining the bottom product stream with an azeotrope breaker component.

The azeotrope breaker component can be effective to improve separation of ethane from the sulfurous material.

The azeotrope breaker component can be predominately carbon dioxide.

The azeotrope breaker component can be provided at a pressure that is greater than an operating pressure of the second contacting column, and wherein the azeotrope breaker component is configured to be predominately in a liquid form at the provided pressure.

The stream exiting the second contacting column and comprising at least ethane further can comprise carbon dioxide.

The method further can comprise processing the stream comprising at least ethane and carbon dioxide in a third contacting column to provide a stream having a carbon dioxide content of at least 90% molar and to provide a stream comprising the ethane.

The method further can comprise adding an azeotrope breaker liquid agent to the third contacting column and recovering at least a portion of the azeotrope breaker liquid agent with the stream comprising the ethane.

The method further can comprise processing the stream comprising the ethane in a fourth contacting column to provide a first stream having an ethane content of at least 90% molar and a second stream comprising an azeotrope breaker blend.

The stream comprising at least a portion of the sulfurous material exiting the second contacting column further can comprise hydrocarbons that are C3 or higher.

The method further can comprise processing the stream comprising at least a portion of the sulfurous material and hydrocarbons that are C3 or higher in a fifth contacting column effective to provide a stream comprising predominately the sulfurous material and to provide a stream comprising the hydrocarbons that are C3 or higher.

The method further can comprise processing the stream comprising the hydrocarbons that are C3 or higher in a sixth contacting column to provide a stream comprising predominately propane and to provide a stream comprising hydrocarbons that are C4 or higher.

Prior to the processing in the first contacting column, the multi-component feed stream including at least a sulfurous material and a fuel gas can be at a pressure greater than ambient pressure.

The method further can comprise expanding the multi-component feed stream in an expansion unit to a pressure of less than 60 bar prior to the processing in the first contacting column and/or to a pressure of about 30 bar to about 49 bar.

The expansion unit can be, for example, a turbo expander.

The expansion unit can be, for example, a compander.

The method further can comprise processing the multi-component feed stream prior to processing in the first contacting column such that a portion of the multi-component feed stream is in a liquid state.

The method further can comprise removing at least part of the portion of the multi-component feed stream that is in a liquid state and further processing said at least part separate from the first contacting column.

### BRIEF DESCRIPTION OF THE DRAWINGS

Having thus described the disclosure in the foregoing general terms, reference will now be made to the accompanying drawings, which are not necessarily drawn to scale, and wherein:

FIG. 1 is a flow diagram showing a system for separation of carbon dioxide and sulfurous material from a multi-component feed stream and the separation of a CO₂/ethane mixture from the resulting residual stream according to embodiments of the present disclosure;

FIG. 2 is a flow diagram showing a system for separation of a carbon dioxide and sulfurous material from a multi-component feed stream according to embodiments of the present disclosure;

FIG. 3A is a partial flow diagram showing components useful for reduction of N₂ content in a final natural gas product according to further embodiments of the present disclosure;

FIG. 3B is a partial flow diagram showing components useful for production of liquefied natural gas according to further embodiments of the present disclosure;

FIG. 3C is a partial flow diagram showing components useful for helium recovery (HeRU) and nitrogen rejection (NR) according to further embodiments of the present disclosure; and

FIG. 3D is a partial flow diagram showing components useful for production of liquefied natural gas and the recovery of helium according to further embodiments of the present disclosure.

### DETAILED DESCRIPTION

The present subject matter will now be described more fully hereinafter with reference to exemplary embodiments thereof. These exemplary embodiments are described so that this disclosure will be thorough and complete, and will fully convey the scope of the subject matter to those skilled in the art. Indeed, the subject matter can be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

The present disclosure provides systems and methods suitable for separation of carbon dioxide and sulfurous materials from mixed gas streams and fractionation of the mixed gas streams into various constituents. More particularly, it has been found that carbon dioxide in its liquid form can be highly effective as a solvent for the absorption of sulfurous materials from a gaseous or liquid stream. As such, liquid carbon dioxide (or a further solvent as defined herein) can be used in some embodiments for removing sulfurous materials from multi-component feed streams that further include one or more fuel gases. This then can provide a highly cost effective means for purifying fuel gases (or other products) by removal of substantially all of the contaminating sulfurous materials that may be present therein.

A multi-component feed stream suitable for treatment according to the present disclosure can comprise at least three components—a gaseous component that is to be purified for provision as a product, carbon dioxide, and a sulfurous material to be separated or removed from the multi-component feed stream. In some embodiments, the gaseous component to be purified can be a fuel gas. For example, methane or a mixture of methane and ethane may be the fuel gas. In such embodiments, the multi-component feed stream may thus be a natural gas mixture that contains methane, ethane, and some content of higher carbon number hydrocarbons (e.g., C₃ to C₆). In some embodiments, the fuel gas may comprise carbon monoxide and/or hydrogen. For example, the fuel gas may be syngas. In some embodiments, the fuel gas may be a petroleum gas, which is typically comprised of saturated linear hydrocarbons with 1 to 5 carbon atoms, carbon dioxide, hydrogen sulfide, and nitrogen. As such, reference herein to a fuel gas can mean any material that is suitable for use as a fuel and that is gaseous under its normal conditions (e.g., at standard temperature and pressure of 0° C. and 1.01 Bar). In some embodiments, the fuel gas may contain a small amount or a significant amount of one or more inert gases, such as nitrogen and/or helium. In some embodiments, a multi-component feed stream can include any one or a combination of the following components: methane, ethane, propane, butane, pentane, hexane, carbon monoxide, and hydrogen. Preferably, the multi-component feed stream comprises at least a gaseous component and thus, the multi-component feed stream may be referred to as a gaseous mixture.

The sulfurous material to be separated or removed from the multi-component feed stream beneficially can be substantially any sulfur-containing material since the solvent utilized according to the present disclosure can be configured to effect removal of substantially all sulfur-containing species from a multi-component feed source. For example, liquid carbon dioxide in particular can be effective to absorb and/or condense substantially all sulfur-containing species from a hydrocarbon mixture. As such, sulfurous materials such as hydrogen sulfide (H₂S), carbonyl sulfide (COS), thiol-containing materials (e.g., mercaptans), carbon disulfide, and disulfide bond-containing compounds may be may be separated or removed from a multi-component feed stream according to embodiments of the present disclosure. It is understood that a thiol-containing material can be any organosulfur compound of the formula R—SH, wherein R is an alkyl or other organic substituent. A thiol-containing material may be referred to as a mercaptan.

In an example embodiment, the present disclosure can relate to treatment of a sour gas, which is understood to be a natural gas or any other gas containing significant amounts of hydrogen sulfide. Sour gas may contain further acidic gases, such as carbon dioxide gas. Although the invention may be discussed herein, in example embodiments, in relation to treatment of a sour gas, it is understood that the disclosure extends to treatment of any multi-component feed stream containing a sulfurous material.

A multi-component feed stream suitable for treatment according to the present disclosure may contain at least 1,000 ppm, at least 1%, at least 2%, or at least 5% of sulfurous materials on a molar basis. Although an upper limitation on the amount of sulfurous materials present in the multi-component feed stream is not required, in some embodiments, any of the foregoing levels may be defined to have an upper limit of 50%, 40%, 30%, or 20% on a molar basis (e.g., encompassing a range of 1,000 ppm to 50% molar, or any intervening ranges).

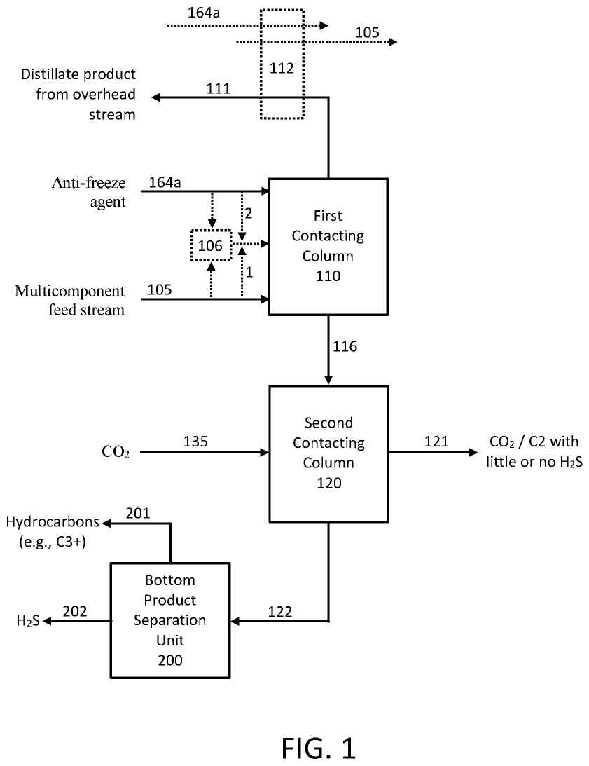

Referring generally to FIG. 1, a multi-component feed stream including at least a sulfurous material, carbon dioxide, and a product gas comprising methane and ethane (e.g., sour natural gas) can be processed to separate at least a portion of the carbon dioxide and the sulfurous material from the product gas. Specifically, the multi-component feed stream can be injected through line 105 into a first contacting column 110 from a source of the multi-component feed stream. As further described below, the multi-component feed stream may be subjected to one or more processing steps prior to injection into the first contacting column 110. For example, the multi-component feed stream may be cooled to a temperature warmer than the freezing point of carbon dioxide. As such, the multi-component feed stream in line 105 injected into the first contacting column 110 may include a liquid fraction as well as a gaseous fraction. As such, the term gaseous mixture may be used to define the multi-component feed stream but should not be viewed as excluding fluid streams comprising as liquid fraction as well as a gaseous fraction since two-phase flow streams can be readily processed according to the present disclosure. For example, in some embodiments, a multi-component feed stream including a liquid fraction and a gaseous fraction may be injected directly into the first contacting column 110 using a gallery tray, chimney tray, and/or similar internal structural components that are commonly utilized for accommodating two-phase injection streams as line 105. As another example, a multi-component feed stream that includes a liquid fraction and a gaseous fraction may be flashed into vapor and liquid fractions prior to injection into first contacting column 110. In further examples, the multi-component feed stream containing a liquid fraction and a gaseous fraction may be separated by fraction with the gaseous fraction being injected into a middle section of the first contacting column 110, and the liquid fraction being injected into another section of the same column or bypassing the first contacting column 110 and being mixed with the liquid bottom stream exiting the contacting column for injection into a second contacting column (e.g., second contacting column 120 in FIG. 1). Thus, while line 105 is illustrated as a single line, it is understood that line 105 may represent a single line or a plurality of lines.

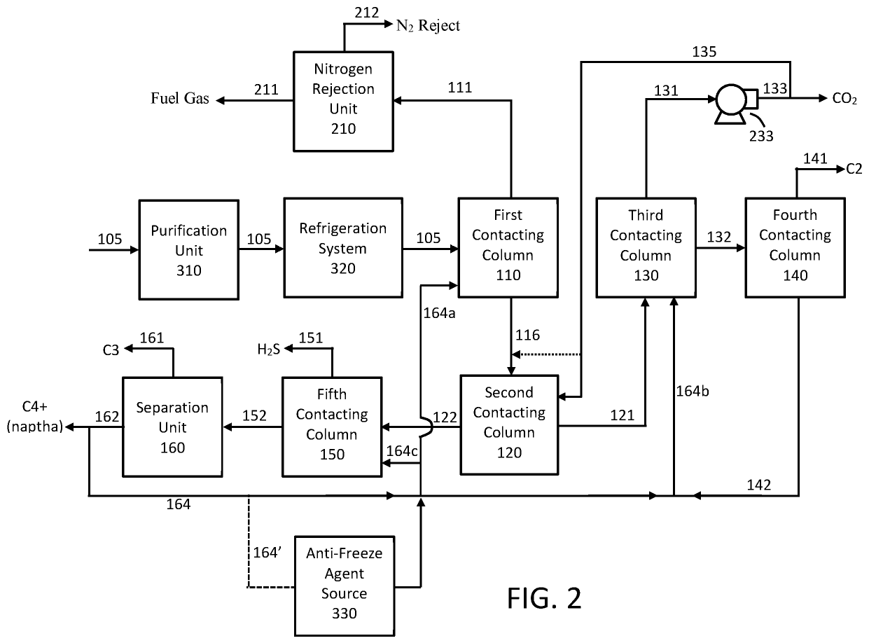

In some embodiments, the multi-component feed stream, prior to injecting into the first contacting column, may be at a pressure greater than ambient. For example, the multi-component feed stream may be at a pressure of about 10 bar to about 200 bar, about 3 bar to about 100 bar, about 4 bar to about 80 bar, or about 5 bar to about 50 bar. In preferred embodiments, lower pressure ranges may be particularly useful, such as ranges of about 10 bar to about 70 bar, about 20 bar to about 60 bar, about 30 bar to about 50 bar, or about 35 bar to about 49 bar. Providing the multi-component feed stream at such pressures can be beneficial to allow for cooling of the stream, and/or to provide power for the process and/or for export, and/or to provide energy for compression of one or more streams that are produced in the process. As such, the present systems can include one or more expanders, for example as a component of unit 310 and/or unit 320 as shown in FIG. 2. In FIG. 2, the multicomponent feed stream is passed through line 105 to the first contacting column 110 while passing through the intervening purification unit 310 and refrigeration system 320. It is understood that purification unit 310 and refrigeration system 320 may both be absent, may both be present, or only one of the purification system 310 or the refrigeration system 320 may be present. In some embodiments, a turbo expander may be utilized. In further embodiments, a compander type unit may be utilized where one or more expanders and one or more compressors are provided on a common shaft. The one or more expanders may be available to expand at least a portion of the multi-component feed stream, and the one or more compressors may be available to compress one or more further streams produced by the present process.

The multi-component feed stream in line 105 preferably is contacted with an anti-freezing agent. The contact between the multi-component feed stream and the anti-freezing agent can be characterized as a reflux or other intimate mixing, including but not limited to providing for a counter-current flow of the multi-component feed stream against the anti-freezing agent. Although the anti-freezing agent is illustrated in FIG. 1 as being injected into the first contacting column 110, it is understood that the anti-freezing agent may be mixed partially or completely with the multicomponent feed stream prior to entry into the first contacting column. For example, lines 105 and 164a may mix prior to entry into the first contacting column 110 (as illustrated in FIG. 1 with arrows 1 and 2). Alternatively, lines 105 and 164a may be input into a mixing unit (106) prior to being passed to the first contacting column 110. The anti-freezing agent is preferably a mixture of hydrocarbons that remain in liquid form at suitable temperatures for use in the first contacting column 110. In particular, the hydrocarbon(s) used as the anti-freezing agent preferably are liquid at temperatures within the range of about −95° C. to about 0° C., about −90° C. to about 0° C., about −80° C. to about −20° C., or about −70° C. to about −30° C. Suitable hydrocarbon(s) can particularly include one or more C3 to C8, C4 to C8, or C4 to C7 compounds and particularly one or more linear hydrocarbon(s) from the group of butanes, pentanes, and hexanes. Such materials can be particularly useful for suppressing solidification of acidic compounds in the first contacting column 110. As further illustrated in FIG. 2, the anti-freezing agent may be taken at least in part from one or more further streams in the present system. Alternatively, or additionally, the anti-freezing agent may be provided from an anti-freezing agent source 330. The anti-freezing agent source 330 may be configured as a storage tank of similar unit where anti-freezing agent may be replenished, which may include directing one or more streams exiting one or more units of the present system to the anti-freezing source for temporary storage.

The multi-component feed stream may comprise at least some content of ethane. For example, the multi-component feed stream may comprise gaseous ethane, such as in an amount of about 1% to about 15%, about 2% to about 10%, or about 3% to about 5%, on a molar basis. As otherwise described herein, the multi-component feed stream may be cooled to a substantially low temperature prior to injecting into the first contacting column 110. At such temperature, some constituents of the multi-component feed stream may be in the liquid form. It is understood, however, that at least a portion of the gases originally present in the multi-component feed stream may remain gaseous. As such, the multi-component feed stream may be in a mixed form wherein a portion of the mixture is liquid and a portion of the mixture is gaseous. In preferred embodiments, a majority (i.e., greater than 50%) of the multi-component feed stream (on a molar basis) remains in a gaseous form when injected into the first contacting column 110.

As discussed herein, cooling the multi-component mixture to a suitable temperature allows an effective phase separation of methane from other constituents of the mixture including ethane and higher hydrocarbons, carbon dioxide, and sulfurous material such as hydrogen sulfide. For example, for a column operating at a pressure of about 40 bar, operating an overhead condenser at approximately −95° C. will result in substantially pure methane distillate product. At these temperatures, however, carbon dioxide forms a solid phase, which is undesirable due to the operation problems such as clogging and blockages of the piping and equipment. It is understood that the use of the anti-freeze agent can suppress the freezing temperature of carbon dioxide, allowing nearly complete separation of methane. The anti-freeze agent introduced to the first contacting column 110 is preferably at a temperature that results in optimum reflux ratio and condenser refrigeration load within the column.

The anti-freeze agent and/or the multi-component feed stream may be subjected to one or more treatments prior to injection into the first contacting column 110 to achieve one or more desired physical conditions for the respective streams. For example, one or both of the anti-freeze agent and the multi-component feed stream may be cooled to the desired temperature range. As illustrated in FIG. 1, the anti-freeze agent and the multi-component feed stream are already at the desired temperature. If cooling is to be applied, however, any refrigeration or other heat exchange suitable to cool one or both streams to the desired temperature range may be used. Example embodiments of cooling units suitable according to the present disclosure are more specifically described in relation to Example 1. In particular, a heat exchange system used for cooling one or both of the anti-freeze agent and the multi-component feed stream may also be utilized to heat the overhead vapor stream in line 111 and/or one or more further gas stream as otherwise discussed herein to a greater temperature, such as to near ambient temperature. When a heat exchanger is used, any deficiency of refrigeration can be provided by a suitable system, such as a closed cycle refrigeration loop with a working fluid capable of providing refrigeration at the required low temperature level. Optionally the refrigeration loop can provide refrigeration at two or more temperature levels. If desired, any refrigeration means known in the art may be utilized. For example, suitable combinations of expansion and recompression may be carried out to provide cooling, and this can encompass systems and methods designed to utilize the Joule-Thomson Effect.

More particularly, prior to injecting the multi-component feed stream in line 105 and/or the anti-freeze agent in line 164a into the first contacting column 110, one or both of these streams may be passed through a heat exchanger against at least a portion of an overhead vapor stream 111 that is withdrawn from the first contacting column 110 so that the overhead vapor stream is heated and one or both of the multi-component feed stream and the anti-freeze agent is cooled. This optional arrangement is illustrated in FIG. 1 in relation to the optional heat exchanger 112. In one or more embodiments, refrigeration for one or both of the multi-component feed stream in line 105 and the anti-freeze agent in line 164a can be provided with a refrigeration system 320 (e.g., a closed cycle loop) using a suitable refrigerant. In this manner, one or more streams of the liquid refrigerant can be evaporated in the heat exchanger (e.g., at the same or at different temperature levels) to maintain a heat balance thereof. In one or more embodiments, the multi-component feed stream may be pre-treated to separate any liquid phase water and optionally hydrocarbons above C4. The so treated multi-component feed stream may then be dried to a dew-point that is below the operating temperature of the first contacting column 110, preferably below a dew point of about −95° C. Such drying may be carried out, for example, in a thermally regenerated desiccant drier. Purification unit 310, for example, can include one or more drying beds, mercury removal beds, and/or similar elements useful for purifying the multi-component feed stream 105. The anti-freeze agent likewise can be provided with a dew-point in one or more of the above-noted ranges. The dried crude multi-component feed stream and the dried anti-freeze agent can then be cooled in the optional heat exchanger 112 (if needed) to the low temperature required for operation of the first contacting column 110. As seen in FIG. 1, the anti-freeze agent and the multi-component feed stream may already have the desired dew-point. Alternatively, referring to the appended Example, the present system and method may include carrying out the desired drying in order to achieve the desired dew-point.

In one or more embodiment, the multi-component feed stream may be partially cooled and separated into gas and liquid fractions and only the gaseous fraction is processed in the first contacting column 110. In such embodiment, the liquid fraction of the partially cooled multi-component stream may be introduced to a different stage of contacting column 110, or be processed in another column within this processes or exported for further processing in another process/facility.

The first contacting column 110 can be any structure that allows for injection of the necessary streams and provides for contacting therein of the injected streams. For example, the first contacting column 110, in some embodiments, may be a distillation column and thus may include any number of trays as may be present in a typical fractionating distillation column Alternatively, the first contacting column 110 may contain any suitable packing material that typically may be utilized in fractionating columns The first contacting column 110 may be characterized as a counter-current contacting column wherein a gaseous mixture passes upward for contacting a liquid mixture that passes downward. The first contacting column 110 preferably may be adapted to or configured to effectively handle a two-phase feed stream. For example, the first contacting column 110 may be adapted to or configured to provide for any one or more of the following: flash a feed stream in a flash vessel prior to the entrance to the column; use a gallery tray or chimney tray; and/or include heating elements such as heating coils or tubing containing heating fluids such as steam or other process streams in internal sections of the column in order to adjust the boil-up within the column and obtain desired separation performance over its top and bottom products. The first contacting column and/or any one or more of all further contacting columns described herein can be configured with any components typically integral with such units or capable of being integrated with such units, such as one or both of an overhead condenser and a bottom reboiler. It is understood that an overhead condenser and a bottom reboiler are internal components of contacting columns and, as such, are not necessary for illustration in the appended figures.

The operating pressure of the first contacting column 110 preferably is below the critical pressure of the fluids in the first contacting column at all points. In example embodiments, the first contacting column can operate at a pressure in the range of about 7 bar to about 55 bar, about 10 bar to about 50 bar, or about 30 bar to about 46 bar. Likewise, the anti-freeze agent and/or the multi-component feed stream may be injected to the first contacting column 110 at a pressure within one or more of the ranges noted above.

In practice, the amount of anti-freeze agent introduced into the first contacting column 110 can be adjusted such that a suitable approach to the freezing point of CO₂ from both liquid and gaseous phase is maintained within all sections of the first contacting column 110.

In one or more embodiment, a distillate product (or overhead stream) exiting the first contacting column 110 through line 111 can have a low sulfur content. For example, the sulfur content of the distillate product in line 111 can be less than 150 ppmv, less than 50 ppmv, less than 10 ppmv, or less than 4 ppmv (e.g., possibly being as low as 0 ppmv). The distillate product, for example, can be a fuel gas a defined herein.

In one or more embodiment, the distillate product in line 111 exiting the first contacting column 110 can have a CO₂ content of less than 5%, and preferably less than 2%, on a molar basis. By controlling the temperature of the overhead condenser section of the contacting column and introducing a suitable amount of the anti-freeze agent into the first contacting column 110, CO₂ content of 50 ppmv or lower can be achieved (possibly as low as 0 ppmv). In particular, if an overhead methane product in line 111 is to be further cooled, overhead CO₂ content of less than 2%, less than 1%, less than 200 ppm, and preferably less than 50 ppm can be achieved. For example, in embodiments wherein the multi-component feed stream contains substantial amount of nitrogen and/or helium that are desired to be removed or recovered, the distillate product in line 111 exiting the first contacting column 110 can be further cooled down and processed to achieve the desired separation. Another example is production of liquefied natural gas (LNG) from the overhead vapor of the first contacting column 110, which requires cooling the overhead vapor product to a temperature of around −163° C. To avoid solidification at such a low temperature, CO₂ content of the overhead vapor should be approximately 50 ppmv or less.

A bottom product stream in line 116 can be withdrawn from a lower portion of the first contacting column 110, the bottom product stream containing at least a portion of the sulfurous material and any carbon dioxide from the multi-component feed stream. As already discussed above, an overhead vapor stream in line 111 can be withdrawn from an upper portion of the first contacting column 110, the overhead vapor stream containing at least a portion of the product gas from the multi-component feed stream. In one or more embodiments, the bottom product stream in line 116 can include substantially all (e.g., at least 97%, at least 98%, at least 99%, at least 99.5%, or at least 99.9% molar) of the sulfurous material from the multi-component feed stream. In embodiments wherein the multi-component feed stream is a natural gas, the bottom product stream in line 116 may likewise include substantially all (e.g., at least 97%, at least 98%, at least 99%, at least 99.5%, or at least 99.9% molar) of the C₂ or higher hydrocarbons that are present as well as substantially all (e.g., at least 97%, at least 98%, at least 99%, at least 99.5%, or at least 99.9% molar) of the carbon dioxide and a minor amount (e.g., less than 5%, less than 3%, or less than 1% molar) of methane from the multi-component feed stream.

In embodiments wherein the multi-component feed stream is a natural gas, the overhead vapor stream in line 111 preferably comprises substantially no hydrocarbons greater than C₁. For example, the overhead vapor stream in line 111 exiting the contacting column 110 preferably includes less than 1% or less than 0.1% of any C₂+ compounds, on a molar basis. More particularly, overhead vapor stream in line 111 preferably comprises less than 1% or less than 0.1% of C2, C3, C4, or C5, on a molar basis, each individually or in total.

In some embodiments, it can be useful to heat the overhead stream, such as to near ambient temperature, and utilize its available refrigeration capacity for cooling other process streams. This is typically the case when overhead vapor in line 111 is the final fuel gas product and does not require carrying out further processing at cryogenic temperatures. This can be done utilizing dedicated heating or utilizing heat available from another unit. For example, the overhead vapor stream in line 111 may be passed through the optional heat exchanger 112 that is utilized for cooling of one or both of the anti-freeze agent stream and the multi-component feed stream. This utilizes heat withdrawn from one or both of the original anti-freeze agent stream and the original multicomponent feed stream in order to heat the overhead vapor stream in line 111.

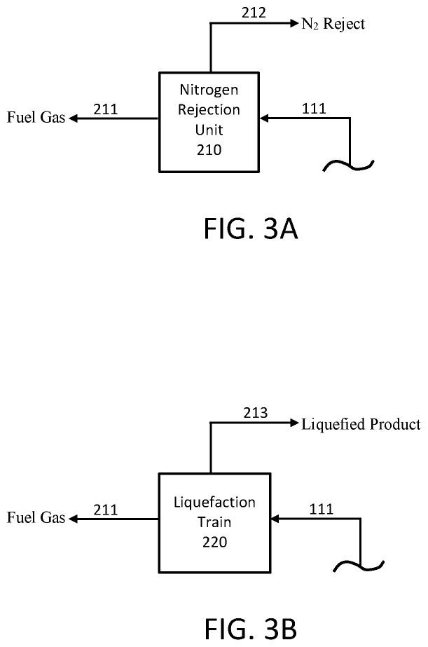

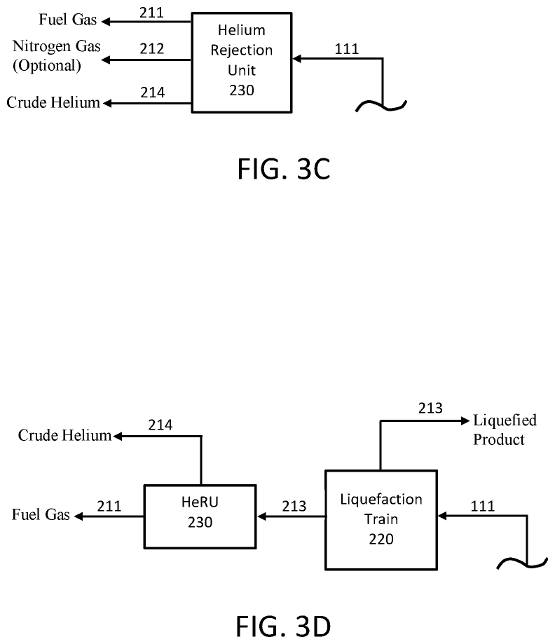

As further illustrated in FIGS. 3A through 3D, the overhead vapor stream in line 111 may be subjected to one or more separation steps in order to separate the vapor stream into desired constituents. For example, when the multicomponent fees stream in line 105 is a natural gas stream, the overhead vapor stream in line 111 may comprise at least methane and one or both of helium and nitrogen. Accordingly, it can be beneficial to separate at least a portion of such material(s) from the methane to meet the minimum required heating value of the fuel gas per applicable standards and regulations. Such concept can be applied equally to any overhead gas stream to the extent that it includes multiple components.

In one or more embodiments, as seen in FIG. 3A, the overhead vapor stream in line 111 can be passed through a nitrogen rejection unit 210 such that a substantially pure (e.g., at least 90%, at least 98%, at least 99%, or at least 99.5% molar nitrogen) stream of nitrogen exits in line 212 and a final fuel gas stream exits in line 211. The fuel gas in line 211 again will have a desired purity level as described herein (e.g., less than 4 ppmv H₂S) and specifically can include less than 5%, less than 4%, or less than 3% molar nitrogen. The nitrogen rejection can be done through variety of methods including distillation, membrane separation, and pressure swing adsorption, and the nitrogen rejection unit 210 thus can comprise any one or more of a distillation unit, membrane separation unit, pressure swing adsorption unit, or other suitable unit effective for nitrogen separation. In particular, the distillation method is very effective for the removal of large nitrogen content of large feed gas flows. Nitrogen has a very low boiling point at approximately −195.8° C., which requires low operating temperature for its separation from methane. Accordingly, if a nitrogen separation step is necessary, it can be beneficial to directly transfer the cold distillate from the first contacting column 110 to the nitrogen rejection unit 210 to reduce the refrigeration energy requirement and capital cost of the processing plant. The nitrogen containing overhead vapor stream leaving the top of the first contacting column 110 through line 111 can be optionally further cooled using an external refrigeration source or another cold stream from the process prior to entering the nitrogen rejection unit 210.

In one or more embodiments, as shown in FIG. 3B, the overhead vapor in line 111 from the first contacting column 110 can be further processed in a liquefaction train 220 to produce a liquefied product, such as liquefied natural gas, in line 213. In such process, the unit forming the liquefaction train can be configured to provide sufficient refrigeration such that the temperature of the feed gas in line 111 can be reduced to the temperature at which methane, the main constituent of natural gas, liquefies (e.g., around −161° C.). If permanent gases, such as nitrogen and/or helium, are available in the feedstock, such gases can be concentrated in the tail gas that is generated as a result of feedstock liquefaction. The present disclosure thus offers significant flexibility for integration with existing LNG processing and allows for the integration of refrigeration systems in both plants. For example, a three-stage cascade refrigeration system can be used to provide the necessary refrigeration duty for sweetening of a starting sour gas, NGL recovery and separation, and LNG production operations. This can substantially reduce the overall cost of the process and improve the energy efficiency, as well as also reducing emissions from the overall process.

In one or more embodiments, it may be beneficial to further process the vapor stream 111 for removal of further constituents. For example, with reference to FIG. 3C, the overhead vapor stream 111 may, in one or more embodiments, contain significant amounts of helium, which beneficially may be recovered for further purification and sales by processing at least a portion of the vapor stream through a helium rejection unit 230. Various process technologies based on adsorption, membrane separation, cryogenic distillation, or the like can be used to recover helium from natural gas. In some embodiments, the present disclosure particularly can be configured to utilize cryogenic distillation helium recovery. The overhead vapor stream 111 can be provided at a relatively low temperature and therefore can be conveniently processed in a cryogenic recovery unit to reduce the overall refrigeration energy that is required. Cryogenic helium recovery is a mature technology and can be carried out is several ways. In embodiments wherein nitrogen content in the multi-component feed stream 105 is sufficiently low, a single column helium recovery design can achieve satisfactory helium recovery rates and produce a helium-depleted fuel gas stream in line 211 and a crude helium stream in line 214. The latter can be further processed using conventional helium upgrading and purification processes. In embodiments wherein a significant content of nitrogen is also available in the multi-component feed stream 105, it will be concentrated in the crude helium stream if a single column helium recovery design is used. This would make the additional helium upgrading and purification process more complex. In such a case, a two-column helium recovery unit may be preferred since it is capable of generating separate fuel gas (in line 211), nitrogen (in line 212), and crude helium (in line 214) product streams.

In some embodiments, the present disclosure can encompass integration of both natural gas liquefaction and helium recovery. For example, as seen in FIG. 3D, the overhead vapor stream 111 may be passed first through a liquefaction train 220 (similarly as described above in relation to FIG. 3B) prior to being passed through a helium rejection unit 230 (similarly to as described above in relation to FIG. 3C). Given that helium is significantly more volatile than methane, initial formation and removal of LNG through line 213 can beneficially be followed by helium recovery (i.e., from line 214 exiting the HeRU) from the tail gas exiting the liquefaction train 220 through line 213.

The bottom product stream exiting the first contacting column 110 in line 116 may be further processed as desired to separate the stream into one or more of its constituents, and such treatment can vary depending upon the composition of the original multi-component feed stream that is introduced to the contacting column 110. As illustrated in FIG. 1, the bottom product stream in line 116 may be further processed in a second contacting column 120, which can be configured to be a De-C2 column. In the De-C2 column 120, a majority of the CO₂ and C2 content from the bottoms product in line 116 can be separated from any C3+ hydrocarbons and sulfurous compounds present in the bottoms product leaving the first contacting column 110. The De-C2 column 120 can be, for example, a distillation column and can be operated at an increased pressure, such as about 10 bar to about 50 bar, about 12 bar to about 40 bar, or about 16 bar to about 35 bar. The De-C2 column 120 likewise may be operated in suitable temperature ranges, such as to produce an overhead vapor stream in line 121 at a temperature of about −40° C. to about 12° C. and to produce a bottoms stream in line 122 at a temperature of about 20° C. to about 75° C.

Processing the bottom product stream in the second contacting column 120 can include combining the bottom product stream with an azeotrope breaker component. The azeotrope breaker can be substantially a single compound or may be a mixture of two or more different compounds. Preferably, the azeotrope breaker component can be a compound or mixture of compounds that is effective to improve separation of ethane and CO₂ from the sulfurous material and C3+ hydrocarbons. For example, the azeotrope breaker component particularly can consist of or comprise carbon dioxide. For example, the azeotrope breaker component may be a substantially pure stream of carbon dioxide (e.g., at least 95%, at least 98%, or at least 99% molar CO₂). In other embodiments, the azeotrope breaker can be predominately carbon dioxide (e.g., greater than 50% carbon dioxide on a molar basis). In certain embodiments, the azeotrope breaker component can be provided at a pressure that is greater than an operating pressure of the second contacting column. For example, the azeotrope breaker component can be provided at a pressure that is at least 1.05, at least 1.1, at least 1.2, or at least 1.5 times greater than the operating pressure of the second contacting column 120 (e.g., up to the acceptable pressure limit of the second contacting column and/or the line through which the azeotrope breaker component is provided. In some embodiments, the azeotrope breaker component can be configured to be predominately in a liquid form at the provided pressure (e.g., at least 51%, at least 60%, at least 75%, at least 90%, at least 95%, or at least 99% by mass liquid). In some embodiments, CO₂ azeotrope breaker stream is refrigerated in a heat exchanger to a suitable temperature to be predominately in a liquid form at the provided pressure.

In the example embodiment shown in FIG. 2, a stream of predominately liquid CO₂ is introduced via line 135 as an azeotrope breaker component to the second contacting column (i.e., the De-C2 column) 120, preferably at a stage close to the bottom of the De-C2 column. The liquid CO_{2,} or another suitable azeotrope breaker component, can be utilized as a solvent to enhance the separation of H₂S and ethane while achieving a high C2 recovery of ethane in the overhead distillate in line 121. This is particularly useful when the relative concentration of ethane to hydrogen sulfide is high in the multicomponent feed stream originally input to the first contacting column 110. Ethane and H₂S form an azeotrope that makes their complete separation by distillation relatively inefficient. By using liquid CO₂ in the De-C2 column 120, a high recovery of ethane can be achieved while the residual H₂S in the distillate can be limited to less than 50 ppmv, less than 10 ppmv, and preferably less than 4 ppmv. The stream introduced through line 135 can be substantially pure, liquid carbon dioxide; however, the stream may have varying purity levels for carbon dioxide. For example, the stream in line 135 can comprise at least 60%, at least 80%, at least 95%, at least 99%, or at least 99.9% on a molar basis liquid carbon dioxide. In particular, it can be beneficial for the stream in line 135 to substantially exclude any sulfurous material (i.e., meaning that the stream in line 135 includes less than 2 ppm molar of any sulfurous material). More preferably, the stream in line 135 can be completely free of any sulfurous material. While carbon dioxide can be particularly useful, the stream in line 135 can include a material that is adapted to or configured to dissolve, condense, or otherwise cause a sulfurous material to separate from further components of the multi-component feed stream and exit the contacting column into which it is injected along with a liquid stream while the remaining portions of the multi-component feed stream exits the contacting column with a gaseous stream. The stream in line 135 can be taken from line 133, downstream from pump 233, or can be taken from line 131, depending upon the desired pressure.

As noted above, it can be beneficial to input the solvent stream, such as liquid CO₂, near the bottom of the De-C2 column 120; however, the solvent stream in line 135 may be introduced in other manners. For example, in some embodiments, the addition of carbon dioxide (or other solvent) to the second contacting column 120 may be done by mixing a predominantly CO₂ containing stream with stream 116 prior entering the second column 120. Alternatively, or additionally, the solvent stream (e.g., liquid CO₂) may be mixed with a reflux stream used in the second contacting column 120.

In some embodiments, the liquid CO₂ introduced into the second distillation column 120 can be replaced with a mixture of hydrocarbons, preferably in the C4-C6 range, to enhance C2 recovery in the distillate stream exiting in line 121. This configuration can be particularly useful when the relative concentration of ethane to both CO₂ and H₂S is low, and the interactions between CO₂ and H₂S is dominant. In such a case, addition of a liquid solvent with high concentrations of linear hydrocarbons can improve the relative vitality between CO₂ and H₂S resulting in a distillate that is concentrated in CO₂ and C2 and depleted in H₂S.

The distillate exiting the De-C2 column 120 through line 121 can be further processed in a first separation unit 130 (also referenced as a third contacting column) and a second separation unit 140 (also referenced as a fourth contacting column) to separate CO₂ and ethane from the distillate. The first separation unit (or third contacting column) 130 can be configured as a distillation column that is typically operated at a pressure of about 10 bar to about 50 bar, about 12 bar to about 40 bar, or about 15 bar to about 35 bar. Likewise, the first separation unit 130 can be configured to provide an overhead stream in line 131 at a temperature of about −28° C. to about 28° C. and a bottoms stream in line 132 at a temperature of about 30° C. to about 85° C. CO₂ and C2 form an azeotrope that makes their complete separation through distillation impossible. To overcome this problem, a liquid agent can be used to improve the volatility between ethane and CO₂ and effectively break the azeotrope. The azeotrope breaker liquid agent is preferably a linear alkane in the C4-C6 range or any combination thereof. As illustrated, the azeotrope breaker is provided to the first separation unit 130 through line 164b. The use of an azeotrope breaker agent can allow for obtaining a high purity (e.g., at least 90%, at least 95%, at least 98%, or at least 99% molar) CO₂ distillate in line 131 exiting the first separation unit 130 and a bottoms stream exiting in line 132 that is mainly comprised of C2 and the azeotrope breaker. As such, the first separation unit 130 effectively functions as a De-CO₂ column. The CO₂ distillate in line 131 can be further processed as needed to provide a CO₂ product stream through line 133. As shown in FIG. 2, the CO₂ distillate can be compressed with pump 233, but the pump may be replaced with one or more compressors or similar components, and combinations of pumps/compressors may be used as needed.

The bottoms stream exiting the first separation unit 130 in line 132 can be further fractionated in the second separation unit 140 to obtain high purity (e.g., at least 90%, at least 95%, at least 98%, or at least 99% molar) C2 as the distillate in line 141. The majority of the azeotrope breaker that was initially input to the first separation unit 130 through line 164b can be obtained as the bottoms product exiting the second separation unit (i.e., the fourth contacting column) 140 in line 142 to be recycled back to the first separation unit 130. The second separation unit 140 can be configured as a distillation column with an operating pressure of about 10 bar to about 50 bar, about 12 bar to about 40 bar, or about 15 bar to about 35 bar. The second separation unit 140 can be configured to provide a distillate stream in line 141 at a temperature of about −20° C. to about 16° C. and to provide a bottoms stream in line 142 at a temperature of about 91° C. to about 140° C.

The bottom product in line 122 exiting the second contacting unit 120 can be further processed to recover one or more of its constituents based on its composition and the desired separation. In some embodiments (e.g., as illustrated in FIG. 1), a bottom product separation unit 200 can be utilized for this purpose and can be adapted to or configured to carry out a Claus process wherein sulfur compounds can be converted into elementary sulfur. In some embodiments, the bottom product separation unit 200 can be adapted to or configured to carry out process steps wherein sulfur compounds are converted into concentrated sulfuric acid. In some embodiments, the bottom product separation unit 200 can be adapted to or configured to carry out sulfur combustion with flue gas desulfurization wherein sulfur compounds are converted into gypsum. In some embodiments, the bottom product separation unit 200 can be adapted to or configured to carry out mineral carbonation storage wherein the carbon dioxide and hydrogen sulfide may be mixed with further compounds.

The separation(s) carried out using the bottom product separation unit 200 can be illustrated using an example wherein the original multi-component feed stream is a sour natural gas stream comprising at least methane, C2 and greater hydrocarbons, gaseous carbon dioxide, and hydrogen sulfide. In such example, the separation sequence can be designed to provide a hydrogen sulfide stream in line 202 that preferably contains less than 1% CO₂ content and also provide a hydrocarbon stream in line 201 (e.g., containing C3 or greater hydrocarbons), preferably with less than 2 ppm hydrogen sulfide content. This example is further discussed below with reference to FIG. 1 and FIG. 2.

In the example embodiments illustrated in FIG. 2, the bottom product stream in line 122 is further processed in a plurality of units that replace the bottom product separation unit 200 shown in FIG. 1. The bottom product stream in line 122 can comprise at least a portion of any hydrogen sulfide and C3 and higher hydrocarbons present in the sour natural gas provided as the multicomponent feed stream. In some embodiments, the bottom product stream in line 122 can contain substantially all of any C3 or greater hydrocarbons, substantially all of the hydrogen sulfide and other sulfurous materials originally present in the natural gas, and small amounts of C2 (ethane) and carbon dioxide. In the illustrated embodiment in FIG. 2, the bottom product stream in line 122 is first passed to a fifth contacting column 150 that can be configured to separate a majority or substantially all of the ethane that is present in the bottom product stream in line 122 and that can be configured to include a reboiler and/or an overhead condenser. The liquid bottom product in line 122 exiting the second contacting column 120 preferably includes substantially no methane—i.e., about 1000 ppm or less, about 500 ppm or less, about 100 ppm or less, or less than 10 ppm on a molar basis. In some embodiments, the stream in line 122 can be at a pressure of about 5 bar to about 35 bar, about 7 bar to about 30 bar, or about 10 bar to about 25 bar. An overhead vapor stream exiting the fifth contacting column 150 in line 151 can comprise substantially all (e.g., at least 90%, at least 98%, at least 99%, at least 99.5%, or at least 99.9% on a molar basis) of the hydrogen sulfide from the original sour natural gas source 202. Removal of the hydrogen sulfide in the fifth contacting column 150 can include adding to the column an azeotrope breaker reflux stream comprising C4 and greater hydrocarbons through line 164c, which can be provided from an external source or optionally can be taken from an exit stream of a further component of the system (such as described below). The addition of the higher hydrocarbon stream to the fifth contacting column 150 allows substantially all of the C3 and greater hydrocarbons from the sour natural gas to be delivered as a liquid bottom stream in line 152 that includes substantially no hydrogen sulfide (e.g., less than 350 ppm on a mass basis). In some embodiments, the overhead vapor stream in line 151 can be enriched in carbon dioxide —e.g., comprising about 0.1% to about 20%, about 0.5% to about 15%, or about 1% to about 10% carbon dioxide on a molar basis.

The bottom product in line 152 exiting the fifth contacting column 150 can be provided into a separation unit 160, which can function substantially as a de-propanizer. A C3 (i.e., propane) stream can be withdrawn in line 161, and the C4 and greater hydrocarbon stream (i.e., a naphtha stream) can be withdrawn in line 162. As illustrated, all or a portion of the naphtha stream (or other mixture of C₄ and greater hydrocarbons) can be taken from line 162 as a product stream. If desired, a portion of the C4 and greater hydrocarbon stream in line 162 can be separated into line 164 as a recycle stream to provide part or all of a reflux stream that can be input to any one or more further units as described herein. For example, all or a portion of the C4 and greater hydrocarbon stream in line 164 may be utilized in any one or more of the following: provided in line 164a into the first contacting column 110; provided in line 164b into the third contacting column 130; and/or provided in line 164c into the fifth contacting column 150. Likewise, all or a part of the stream from line 164 may be directed through line 164′ to the anti-freeze agent source 330 for temporary storage as needed. The presently disclosed system thus can provide for highly efficient separation of hydrogen sulfide, carbon dioxide, and light hydrocarbon streams (e.g., liquefied petroleum gas (LPG), which can comprise mainly propane, mainly butane, or a mixture thereof) from a sour natural gas feed stream with low capital cost, low utility consumption (and thus low operating cost), and zero emission of carbon dioxide to the atmosphere.

In one or more embodiments, it may be desirable to reduce the concentration of non-H₂S sulfurous materials in the C3+ product (i.e., the stream delivered through line 152). The current system is capable of separating all type of sulfurous materials such as mercaptans, carbon disulfide, carbonyl sulfide, and hydrogen sulfide that are commonly found in sour natural gas and associated oil gas to provide the fuel gas stream in line 111 with low or no concentration of such materials. Consequently, these compounds will be blended with higher hydrocarbons (C3+) in line 122 because of their relatively higher boiling point. As these compounds enter the fifth contacting column 150, they may partially or completely be removed from the hydrocarbon stream leaving the bottom of this column in steam 152.

The efficacy of the removal of non-H₂S sulfurous materials in the column 150 as the distillate product is mainly a function of the boiling point of such compound. If the boiling point of such compound(s) is lower or close to that of H₂S, all or a large portion of such compound(s) can be separated with the H₂S stream, otherwise at least a portion of such compound would leave the columns mixed with bottoms hydrocarbons. It may be desirable to reduce the total sulfur content of the C3+ blend that is provided in line 152 to less than 1000 ppmw, less than 500, less than 350, or preferably less than 100 ppmw. In such case, an additional processing may be needed to reduce the sulfur content of stream 152 prior to entering the sixth contacting column 160. Such processing may be done in one or more unit operations.

Such steps may include, for example, sulfur removal adsorbent beds, caustic wash, or other method than can be effective at removing specific sulfur species from liquefied hydrocarbon streams. Alternatively, final sulfur polishing of stream 152 may be carried out on only one of the distillate or bottoms product from separation unit 160. For example, if the dominate non-H₂S component in line 152 is COS, it is expected that almost all of COS will leave the separation unit 160 blended with distillate product (C3 or LPG product). In such case, the polishing step will be preferably applied only to the distillate product stream from the column 160.

The presently disclosed systems and methods can be utilized generally to process a multi-component feed stream that includes one or more components that have a desired end use (e.g., a fuel gas, such as a C1 hydrocarbon or a C1 hydrocarbon and a mixture of one or more hydrocarbons of C2 and greater) and one or more compounds that typically considered contaminants (e.g., a sulfurous material) to provide a cleaned product gas and a stream comprising at least the contaminant. The latter stream can be processed in a variety of manners to provide further products. Non-limiting examples of the further processing are provided below.

In one or more embodiments, the present disclosure can encompass systems and methods wherein a multi-component feed stream including a sulfurous material is contacted with one or more solvents effective to separate the sulfurous material. A sulfurous compound that is separated from the multi-component feed stream can be converted to, for example, elemental sulfur. In further embodiments, a sulfurous material that is separated from the multi-component feed stream can comprise hydrogen sulfide, and such material can be converted to sulfuric acid.

In one or more embodiments, the present disclosure can encompass systems and methods wherein a multi-component feed stream including a sulfurous material is contacted with one or more solvents effective to separate the sulfurous material. Hydrogen sulfide that is separated in this manner maybe converted to hydrogen gas and one or more further byproducts. This conversion can be carried out utilizing a variety of methods. For example, such conversion can include any one or more of thermal conversion, thermo-catalytic conversion, electrolysis, and/or thermochemical cycles (e.g., an iodine cycle). As a non-limiting example, provided in Table 1 and Table 2 below are example embodiments of processing conditions for a process carried out utilizing a configuration as shown in FIG. 2. The provided values are to show an example of processing conditions for a single, example embodiment as an illustration of conditions that may be utilized in a working embodiment. Such conditions should not be construed as being the only workable embodiment of the present disclosure, and it is understood that the illustrated values may change when the systems and methods as described herein are carried out under further conditions that would be recognized as workable by the person of skill in the art having the benefit of the present disclosure.

TABLE 1

| | | | | | | | |
|---|---|---|---|---|---|---|---|
| Stream/Line | 105 | 111 | Fuel gas | 116 | 122 | 151 | 152 |
| Flowrate | 4980.4 | 3436.7 | 3170.6 | 21.93.7 | 1580.7 | 696.0 | 1346.7 |
| (kmol/hr) | | | | | | | |
| Temperature | 30 | −89.4 | 23.9 | 49.9 | 41.3 | 9.0 | 95.0 |
| (° C.) | | | | | | | |
| Pressure (bara) | 45 | 41.2 | 33.3 | 41.9 | 20.2 | 16.5 | 16.8 |
| Composition | | | | | | | |
| Mole fraction | | | | | | | |
| C1 | 0.63 | 0.918 | 0.9895 | 0.0001 | 0 | 0 | 0 |
| C2 | 0.07 | 0 | 0 | 0.1589 | 0.0214 | 0.0485 | 0 |
| C3 | 0.035 | 4.4E−05 | 4.7E−06 | 0.0816 | 0.1133 | 0.0146 | 0.1301 |
| nC4 | 0.005 | 2.1E−05 | 2.3E−05 | 0.1104 | 0.1532 | 0.0006 | 0.2914 |
| iC4 | 0.005 | 2.6E−5 | 2.8E−05 | 0.0992 | 0.1376 | 0.0045 | 0.2634 |
| C5 | 0.005 | 1.9E−05 | 2.0E−05 | 0.0993 | 0.1379 | 0.0005 | 0.2588 |
| C6 | 0 | 0 | 0 | 0.0191 | 0.0266 | 0 | 0.0562 |
| CO2 | 0.06 | 5E−05 | 5.4E−05 | 0.1361 | 0.0005 | 0.0011 | 0 |
| H₂S | 0.13 | 0 | 0 | 0.2951 | 0.4096 | 0.9302 | 1E−05 |
| H₂O | 0 | 0 | 0 | 0 | 0 | 0 | 0 |
| N₂ | 0.06 | 0.087 | 0.01 | 0 | 0 | 0 | 0 |

TABLE 2

| | | | | | | |
|---|---|---|---|---|---|---|
| Stream | 161 | 163 | 121 | 131 | 132 | 141 |
| Flowrate | 175.0 | 1171.7 | 943.0 | 627 | 898.0 | 316.8 |
| (kmol/hr) | | | | | | |
| Temperature | 33.4 | 85.1 | −26.8 | −22.7 | 37.2 | −14.9 |
| (° C.) | | | | | | |
| Pressure (bara) | 11.3 | 11.8 | 19.5 | 18.5 | 18.8 | 17.5 |
| Composition | | | | | | |
| Mole fraction | | | | | | |
| C1 | 0 | 0 | 0.0002 | 0.0003 | 0 | 0 |
| C2 | 0 | 0 | 0.3339 | 0.0361 | 0.3254 | 0.9179 |
| C3 | 0.9514 | 0.0075 | 0 | 0.0004 | 0.0045 | 6.4E−05 |
| nC4 | 0.0042 | 0.3343 | 0 | 0.0008 | 0.2159 | 0 |
| iC4 | 0.0405 | 0.2967 | 0 | 0.0016 | 0.1928 | 0 |
| C5 | 0.0038 | 0.2969 | 0 | 0.0007 | 0.1946 | 0 |
| C6 | 0 | 0.0646 | 0 | 0 | 0.0379 | 0 |
| CO2 | 0 | 0 | 0.6659 | 0.96 | 0.02898 | 0.0820 |
| H₂S | 7.7E−05 | 0 | 1.7E−06 | 0 | 1.8E−06 | 5.E−06 |
| H₂O | 0 | | 0 | 0 | 0 | 0 |
| N₂ | 0 | | 0 | 0 | 0 | 0 |

The terms “about” or “substantially” as used herein can indicate that certain recited values or conditions are intended to be read as encompassing the expressly recited value or condition and also values that are relatively close thereto or conditions that are relatively similar thereto. For example, a value of “about” a certain number or “substantially” as certain value can indicate the specific number or value as well as numbers or values that vary therefrom (+ or −) by 5% or less, 4% or less, 3% or less, 2% or less, or 1% or less. In some embodiments, the values may be defined as being express and, as such, the term “about” or “substantially” (and thus the noted variances) may be excluded from the express value. As a further example, a condition that is “substantially” met can indicate that the exact condition is met or that the condition varies from the stated condition by only an insignificant amount that would be expected to occur due to natural variances.

Many modifications and other embodiments of the inventions set forth herein will come to mind to one skilled in the art to which these inventions pertain having the benefit of the teachings presented in the foregoing descriptions. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. A method for separating a sulfurous material from a multi-component feed stream, the method comprising:
processing within a first contacting column a combination of: 1) a multi-component feed stream including at least a sulfurous material and a fuel gas; and 2) an anti-freeze agent that is liquid at temperatures within a range of about −95° C. to about 0° C.;
removing from the first contacting column an overhead vapor stream containing at least a portion of the fuel gas and removing from the first contacting column a bottom product stream containing at least a portion of the sulfurous material; and
processing the bottom product stream in a second contacting column to provide a stream comprising at least ethane and to provide a separate stream comprising at least a portion of the sulfurous material.

2. The method of claim 1, further comprising processing at least a portion of the overhead vapor stream exiting the first contacting column through a unit configured to separate nitrogen from the fuel gas.

3. The method of claim 1, further comprising processing at least a portion of the overhead vapor stream exiting the first contacting column through a liquefaction train configured to liquefy at least a portion of the fuel gas and form a liquefied natural gas stream.

4. The method of claim 1, further comprising processing at least a portion of the overhead vapor stream exiting the first contacting column through a unit configured to separate helium from the fuel gas.

5. The method of claim 1, wherein processing the bottom product stream in the second contacting column includes combining the bottom product stream with an azeotrope breaker component.

6. The method of claim 5, wherein the azeotrope breaker component is effective to improve separation of ethane from the sulfurous material.

7. The method of claim 5, wherein the azeotrope breaker component is predominately carbon dioxide.

8. The method of claim 5, wherein the azeotrope breaker component is provided at a pressure that is greater than an operating pressure of the second contacting column, and wherein the azeotrope breaker component is configured to be predominately in a liquid form at the provided pressure.

9. The method of claim 1, wherein the stream exiting the second contacting column and comprising at least ethane further comprises carbon dioxide.

10. The method of claim 9, further comprising processing the stream comprising at least ethane and carbon dioxide in a third contacting column to provide a stream having a carbon dioxide content of at least 90% molar and to provide a stream comprising the ethane.

11. The method of claim 10, further comprising adding an azeotrope breaker liquid agent to the third contacting column and recovering at least a portion of the azeotrope breaker liquid agent with the stream comprising the ethane.

12. The method of claim 10, further comprising processing the stream comprising the ethane in a fourth contacting column to provide a first stream having an ethane content of at least 90% molar and a second stream comprising an azeotrope breaker blend.

13. The method of claim 1, wherein the stream comprising at least a portion of the sulfurous material exiting the second contacting column further comprises hydrocarbons that are C3 or higher.

14. The method of claim 13, further comprising processing the stream comprising at least a portion of the sulfurous material and hydrocarbons that are C3 or higher in a fifth contacting column effective to provide a stream comprising predominately the sulfurous material and to provide a stream comprising the hydrocarbons that are C3 or higher.

15. The method of claim 14, further comprising processing the stream comprising the hydrocarbons that are C3 or higher in a sixth contacting column to provide a stream comprising predominately propane and to provide a stream comprising hydrocarbons that are C4 or higher.

16. The method of claim 1, wherein prior to the processing in the first contacting column, the multi-component feed stream including at least a sulfurous material and a fuel gas is at a pressure greater than ambient pressure.

17. The method of claim 16, further comprising expanding the multi-component feed stream in an expansion unit to a pressure of less than 60 bar prior to the processing in the first contacting column.

18. The method of claim 17, comprising expanding the multi-component feed stream to a pressure of about 30 bar to about 49 bar.

19. The method of claim 17, wherein the expansion unit is a turbo expander or is compander.

20. (canceled)

21. The method of claim 1, further comprising one or both of the following:
processing the multi-component feed stream prior to processing in the first contacting column such that a portion of the multi-component feed stream is in a liquid state;
removing at least part of the portion of the multi-component feed stream that is in a liquid state and further processing said at least part separate from the first contacting column.

22. (canceled)
